(19) United States
(12) Patent Application Publication
(10) Pub. No.: US 2026/0152465 A1
(43) Pub. Date: Jun. 4, 2026

(54) METHOD OF PURIFYING ETHYLENEDIAMINE

(71) Applicant: ORIENTAL UNION CHEMICAL CORPORATION, Taipei City (TW)

(72) Inventors: Tun Hung CHENG, Kaohsiung City (TW); Chien Chang CHIANG, Kaohsiung City (TW); Yu Chuan HSU, Kaohsiung City (TW); Wen Yung LIN, Kaohsiung City (TW); Taufik POERNOMO, Kaohsiung City (TW); Yi Tsung KO, Kaohsiung City (TW); Hsi Chin TSAI, Taipei City (TW)

(21) Appl. No.: 19/026,457

(22) Filed: Jan. 17, 2025

(30) Foreign Application Priority Data
Dec. 4, 2024 (TW) 113147007

(51) Int. Cl. C07C 209/84; B01D 3/36; C07C 209/16

(52) U.S. Cl. CPC C07C 209/84; B01D 3/36; C07C 209/16

(57) ABSTRACT

A method of purifying ethylenediamine includes the steps as follows. A phenyl compound is added into an ethylenediamine mixture solution, so as to obtain a mixture to be distilled, wherein the ethylenediamine mixture solution includes at least one of a first organic amine compound with a boiling point higher than ethylenediamine and a second organic amine compound with a boiling point lower than ethylenediamine, and the phenyl compound, ethylenediamine and water form an azeotrope. The mixture to be distilled is distilled under a distillation pressure, so as to obtain a low boiling point mixture, wherein the distillation pressure equals to or is smaller than a normal pressure. The low boiling point mixture is phase-separated and water is removed, so as to obtain a purified ethylenediamine product. A purity of the purified ethylenediamine product is larger than 99.5%.

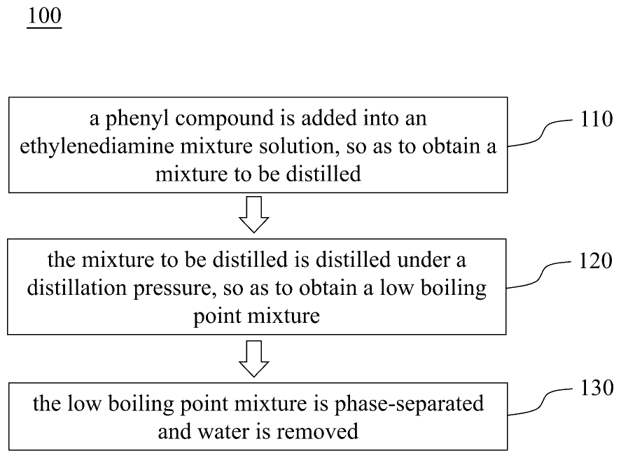

### RELATED APPLICATIONS

This application claims priority to Taiwan Application Serial Number 113147007, filed Dec. 4, 2024, which is herein incorporated by reference.

### BACKGROUND

### Technical Field

The present disclosure relates to a method of purifying amine. More particularly, the present disclosure relates to a method of purifying ethylenediamine using a phenyl compound.

### Description of Related Art

Ethylenediamine is widely used in the production of various products, such as pharmaceuticals, pesticides, lubricants, emulsifiers, bleaching agents, polyamides and epoxy resin curing agents. The primary production methods of ethylenediamine can be categorized into the ethylene dichloride method and the monoethanolamine method. The ethylene dichloride method involves the reaction of ethylene dichloride with ammonia. The monoethanolamine method involves the reductive amination of monoethanolamine with ammonia under a hydrogen atmosphere. However, both the aforementioned two production methods produce various by-products, and it is challenging to obtain high-purity ethylenediamine through conventional purification and distillation processes. For most industrial applications, a minimum purity of 99.5% ethylenediamine is required, so developing advanced purification and separation technologies for ethylenediamine is pretty important.

However, in the conventional methods for separating ethylenediamine and other substances, the ratio of ethylenediamine and other substances is strictly limited, and multiple purification steps are required to possibly obtain ethylenediamine with high purity. For example, when separating ethylenediamine and water, the azeotropic properties of water with other substances can be utilized to remove water. However, this method generates significant amounts of ammonia-nitrogen waste water, and the ratio of water should be strictly controlled. Excess water reduces the yield of ethylenediamine, while insufficient water decreases the purity of ethylenediamine. Alternatively, azeotropic agents which form azeotropes with water can be used for purification, but this method cannot separate ethylenediamine and other substances. Additional purification steps are required, and it is difficult to obtain high-purity ethylenediamine.

In this regard, how to purify ethylenediamine under the conditions of reducing waste water generation and simplifying the production process, and ensuring that the purity and yield of ethylenediamine meet the requirements, has become the goal which the industry strives for.

### SUMMARY

According to the present disclosure, a method of purifying ethylenediamine includes the steps as follows. A phenyl compound is added into an ethylenediamine mixture solution, so as to obtain a mixture to be distilled, wherein the ethylenediamine mixture solution includes at least one of a first organic amine compound with a boiling point higher than ethylenediamine and a second organic amine compound with a boiling point lower than ethylenediamine, and the phenyl compound, ethylenediamine and water form an azeotrope. The mixture to be distilled is distilled under a distillation pressure, so as to obtain a low boiling point mixture, wherein the distillation pressure equals to or is smaller than a normal pressure. The low boiling point mixture is phase-separated and water is removed, so as to obtain a purified ethylenediamine product. A purity of the purified ethylenediamine product is larger than 99.5%.

### BRIEF DESCRIPTION OF THE DRAWINGS

The present disclosure can be more fully understood by reading the following detailed description of the embodiment, with reference made to the accompanying drawings as follows:

The FIGURE is a step flow chart of a method of purifying ethylenediamine according to an embodiment of the present disclosure.

### DETAILED DESCRIPTION

The present disclosure will be further exemplified by the following specific embodiments. However, the embodiments can be applied to various inventive concepts and can be embodied in various specific ranges. The specific embodiments are only for the purposes of description, and are not limited to these practical details thereof.

Referring to the FIGURE, the FIGURE is a step flow chart of a method of purifying ethylenediamine 100 according to an embodiment of the present disclosure. The method of purifying ethylenediamine 100 includes Step 110, Step 120 and Step 130.

In Step 110, a phenyl compound is added into an ethylenediamine mixture solution, so as to obtain a mixture to be distilled. After the phenyl compound is added, the mixture to be distilled can be mixed by stirring, and the mixture to be distilled forms separations. The ethylenediamine mixture solution includes at least one of a first organic amine compound with a boiling point higher than ethylenediamine and a second organic amine compound with a boiling point lower than ethylenediamine. The ethylenediamine mixture solution can be obtained from a reaction between ethylene dichloride (EDC) and ammonia, a reaction between monoethanolamine (MEA) and ammonia or a hydrogenation of cyanamide.

A ratio of ethylenediamine in the ethylenediamine mixture solution can be 10% to 92%. Moreover, the ratio of ethylenediamine in the ethylenediamine mixture solution can be 40% to 80%. Moreover, the ratio of ethylenediamine in the ethylenediamine mixture solution can be 60% to 70%. The aforementioned ratios are weight ratios. Therefore, the ethylenediamine mixture solutions with different ratios of ethylenediamine can be effectively purified by the method of purifying ethylenediamine 100 of the present disclosure.

A weight ratio of ethylenediamine and water in the ethylenediamine mixture solution can be 0.2 to 12. Moreover, the weight ratio of ethylenediamine and water in the ethylenediamine mixture solution can be 0.5 to 5. Moreover, the weight ratio of ethylenediamine and water in the ethylenediamine mixture solution can be 2 to 4. Therefore, the ethylenediamine mixture solutions with different composition ratios can be purified by the method of purifying ethylenediamine 100 of the present disclosure.

The phenyl compound, ethylenediamine and water can form an azeotrope. The target of adding the phenyl compound is to separate ethylenediamine and other substances in the ethylenediamine mixture solution. Therefore, in Step 110, a single type of phenyl compound can be added, or two or more types of phenyl compounds can be added, and the present disclosure is not limited thereto. The phenyl compound can be benzene or toluene, and the phenyl compound can be toluene.

An addition amount of the phenyl compound can be determined by an amount of ethylenediamine in the ethylenediamine mixture solution. The addition amount of the phenyl compound can be 1 time to 40 times of a weight of ethylenediamine in the ethylenediamine mixture solution. Moreover, the addition amount of the phenyl compound can be 1 time to 15 times of the weight of ethylenediamine in the ethylenediamine mixture solution. Moreover, the addition amount of the phenyl compound can be 2 times to 7 times of the weight of ethylenediamine in the ethylenediamine mixture solution. It should be mentioned that, in the method of purifying ethylenediamine 100 of the present disclosure, no matter how much the addition amount of the phenyl compound is, the purified effects can be achieved and the purity of ethylenediamine will not be affected. However, when less phenyl compound is added, the yield of separating ethylenediamine is lower. When more phenyl compound is added, too much phenyl compound will stay at the bottom of the distillation column and should be recycled by additional distillation. Therefore, the manufacturing cost, yield of ethylenediamine and recycling of the phenyl compound should be considered to further adjust the addition amount of the phenyl compound.

Furthermore, before the phenyl compound is added into the ethylenediamine mixture solution, the ethylenediamine mixture solution can be distilled under a reduced pressure, so as to remove the compounds with high boiling points in the ethylenediamine mixture solution. However, even the distillation under the reduced pressure does not perform, the method of purifying ethylenediamine 100 of the present disclosure can achieve great purification effects. Therefore, the present disclosure is not limited thereto.

In Step 120, the mixture to be distilled is distilled under a distillation pressure, so as to obtain a low boiling point mixture. The low boiling point mixture can include the phenyl compound, ethylenediamine and water, and can also include small amount of other substances. The low boiling point mixture can form separations under a normal temperature and a normal pressure. Furthermore, after the mixture to be distilled is distilled, a high boiling point mixture can also be obtained, which can include other substances and small amount of phenyl compound. It should be mentioned that, when the distillation is performed by the distillation column, the low boiling point mixture can be obtained from the top of the distillation column, and the high boiling point mixture can be obtained from the bottom of the distillation column.

The aforementioned distillation pressure equals to or is smaller than the normal pressure, and can equal to the normal pressure. A boiling point of the low boiling point mixture can be 50° C. to 200° C., wherein the boiling point can be taken as the temperature of the top of the distillation column. Moreover, the boiling point of the low boiling point mixture can be 80° C. to 120° C. Moreover, the boiling point of the low boiling point mixture can be 100° C. to 110° C. Therefore, the distillation pressure and temperature can be adjusted according to the composition and ratio of the ethylenediamine mixture solution and the type and addition amount of the phenyl compound, so as to obtain the best distillation effect.

In Step 130, the low boiling point mixture is phase-separated and water is removed, so as to obtain a purified ethylenediamine product, and a purity of the purified ethylenediamine product is larger than 99.5%. In detail, after the low boiling point mixture is phase-separated, an organic phase mixture and an aqueous phase mixture can be separated. The organic phase mixture includes the phenyl compound, and the phenyl compound can be recycled for reuse. The aqueous phase mixture includes ethylenediamine and water. The purified ethylenediamine product can be obtained by removing water in the aqueous phase mixture.

The present disclosure will be further exemplified by the following specific embodiments so as to facilitate utilizing and practicing the present disclosure completely by the people skilled in the art without over-interpreting and over-experimenting. However, the readers should understand that the present disclosure should not be limited to these practical details thereof, that is, these practical details are used to describe how to implement the materials and methods of the present disclosure and are not necessary.

### 1st Example

The product of the reaction between monoethanolamine and liquid ammonia is first distilled to separate the compounds with high boiling points, and an ethylenediamine mixture solution with the composition of 32% of water, 67.64% of ethylenediamine and 0.36% of other substances is obtained.

Next, toluene is added. An addition amount of toluene is 5 times of a weight of ethylenediamine in the ethylenediamine mixture solution. After the mixture is evenly mixed, the mixture is distilled under a normal pressure and a temperature of the top of the distillation column of 103° C. to 110° C., to obtain a low boiling point mixture and a high boiling point mixture. The high boiling point mixture includes small amount of toluene and at least one of the first organic amine compound and the second organic amine compound. The composition of the low boiling point mixture is 8.17% of water, 80.8% of toluene, 10.97% of ethylenediamine and 0.06% of other substances, and the low boiling point mixture forms obvious separations.

Next, the low boiling point mixture is phase-separated under a normal temperature and the normal pressure. After the separation, the upper layer is an organic phase mixture, of which the composition includes toluene with a purity larger than 99.4%. The bottom layer is an aqueous phase mixture, of which the composition is 43.25% of water, 56.61% of ethylenediamine, 0.12% of other substances and 0.02% of toluene. After the water in the aqueous phase mixture is removed, the purified ethylenediamine product with a purity larger than 99.7% can be obtained.

### 2nd Example

The composition of the ethylenediamine mixture solution of the 2nd example is 8% of water, 90.5% of ethylenediamine and 1.5% of other substances. Toluene is added into the aforementioned ethylenediamine mixture solution. An addition amount of toluene is 3 times of a weight of ethylenediamine in the ethylenediamine mixture solution.

After evenly mixing, the ethylenediamine mixture solution is distilled under a normal pressure and a temperature of the top of the distillation column of 103° C. to 110° C., to obtain a low boiling point mixture, of which the composition is 3.53% of water, 74.23% of toluene, 21.97% of ethylenediamine and 0.27% of other substances, with obvious separations.

Next, the low boiling point mixture is phase-separated under a normal temperature and the normal pressure. After the separation, the upper layer is an organic phase mixture, and the bottom layer is an aqueous phase mixture. The composition of the aqueous phase mixture is 21.37% of water, 78.07% of ethylenediamine, 0.55% of other substances and 0.01% of toluene. After the water in the aqueous phase mixture is removed, the purified ethylenediamine product with a purity larger than 99.5% can be obtained.

In summary, according to the method of purifying ethylenediamine of the present disclosure, by adding the phenyl compound and through specific purification steps, there is no need to carry out azeotropic distillation of other substances and water, and large amount of high-concentration ammonia nitrogen waste water will not be produced. Also, there is no need to remove water by pressurizing or distilling with the addition of azeotropic agents, which can significantly reduce waste generation and is favorable for simplifying the purification process and reducing the resource consumption.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method of purifying ethylenediamine, comprising:
adding a phenyl compound into an ethylenediamine mixture solution, so as to obtain a mixture to be distilled, wherein the ethylenediamine mixture solution comprises at least one of a first organic amine compound with a boiling point higher than ethylenediamine and a second organic amine compound with a boiling point lower than ethylenediamine, and the phenyl compound, ethylenediamine and water form an azeotrope;
distilling the mixture to be distilled under a distillation pressure, so as to obtain a low boiling point mixture, wherein the distillation pressure equals to or is smaller than a normal pressure; and
phase-separating the low boiling point mixture and water-removing, so as to obtain a purified ethylenediamine product;
wherein a purity of the purified ethylenediamine product is larger than 99.5%.

2. The method of purifying ethylenediamine of claim 1, wherein the ethylenediamine mixture solution is obtained from a reaction between ethylene dichloride and ammonia, a reaction between monoethanolamine and ammonia or a hydrogenation of cyanamide.

3. The method of purifying ethylenediamine of claim 1, wherein the phenyl compound is benzene or toluene.

4. The method of purifying ethylenediamine of claim 3, wherein the phenyl compound is toluene.

5. The method of purifying ethylenediamine of claim 1, wherein an addition amount of the phenyl compound is 1 time to 40 times of a weight of ethylenediamine in the ethylenediamine mixture solution.

6. The method of purifying ethylenediamine of claim 5, wherein the addition amount of the phenyl compound is 1 time to 15 times of the weight of ethylenediamine in the ethylenediamine mixture solution.

7. The method of purifying ethylenediamine of claim 6, wherein the addition amount of the phenyl compound is 2 times to 7 times of the weight of ethylenediamine in the ethylenediamine mixture solution.

8. The method of purifying ethylenediamine of claim 1, wherein a ratio of ethylenediamine in the ethylenediamine mixture solution is 10% to 92%.

9. The method of purifying ethylenediamine of claim 8, wherein the ratio of ethylenediamine in the ethylenediamine mixture solution is 40% to 80%.

10. The method of purifying ethylenediamine of claim 9, wherein the ratio of ethylenediamine in the ethylenediamine mixture solution is 60% to 70%.

11. The method of purifying ethylenediamine of claim 1, wherein a weight ratio of ethylenediamine and water in the ethylenediamine mixture solution is 0.2 to 12.

12. The method of purifying ethylenediamine of claim 11, wherein the weight ratio of ethylenediamine and water in the ethylenediamine mixture solution is 0.5 to 5.

13. The method of purifying ethylenediamine of claim 12, wherein the weight ratio of ethylenediamine and water in the ethylenediamine mixture solution is 2 to 4.

14. The method of purifying ethylenediamine of claim 1, wherein a boiling point of the low boiling point mixture is 50°C to 200°C.

15. The method of purifying ethylenediamine of claim 14, wherein the boiling point of the low boiling point mixture is 80°C to 120°C.

16. The method of purifying ethylenediamine of claim 15, wherein the boiling point of the low boiling point mixture is 100°C to 110°C.

17. The method of purifying ethylenediamine of claim 1, wherein the distillation pressure equals to the normal pressure.

18. The method of purifying ethylenediamine of claim 1, wherein the low boiling point mixture comprises the phenyl compound, ethylenediamine and water, and the low boiling point mixture forms separations under a normal temperature and the normal pressure.
